(19) United States
(12) Patent Application Publication
(10) Pub. No.: US 2009/0235372 A1
(43) Pub. Date: Sep. 17, 2009

(54) Hairless Immunodeficient Mouse Model

(76) Inventors: Charles B. Clifford, Madison, WI (US); Bruce J. Elder, Andover, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/047,948

(22) Filed: Mar. 13, 2008

(51) Int. Cl. A01K 67/027; C12N 15/01

(52) U.S. Cl. 800/18; 800/22

(57) ABSTRACT

The invention is directed to a hairless, immunodeficient mouse that is homozygous recessive for a hairless (Hr^{hr}) allele and both B-cell deficient and T-cell deficient. The B-cell and T-cell deficiency is attributed to a homozygous recessive scid allele (Prkdc^{scid}), a homozygous recessive beige allele (Lyst^{bg}), or a mutated Rag allele. These mice offer an advantage over nude mouse models used in cancer research because they can be maintained as homozygous breeding pairs and they have greater immune deficits, which permit better growth of xenogenic tumor lines, and rapid assessment of cutaneous and subcutaneous tumors and tissue grafts.

### FIELD OF INVENTION

The field of the invention is medical research, immunology, and oncology, and, in particular, the development of a mouse model.

### BACKGROUND

Immunodeficient mouse models are useful in medical research, particularly oncology and immunology research, because an immunodeficient recipient mouse can receive foreign tissue and tumor transplants, called xenografts, without eliciting an immune response rejecting the foreign tissue or tumor. The immunodeficient mouse model most commonly used in xenograft studies is the athymic nude mouse. Nude mice homozygous for a recessive Foxnl^{nu} mutation are T-cell deficient (thus are immunodeficient) and lack a hair coat. Nude mice heterozygous for the Foxnl^{nu} mutation, however, are immunocompetent and have a normal coat of hair. Another commonly used immunodeficient mouse model is known as the NIH III mouse model, in which the mice are T-cell, B-cell, and natural killer cell deficient.

The breeding of nude mice presents certain challenges. Homozygous female nude mice cannot be used for breeding because of poor reproductive performance due to an irregular estrous cycle and limited lactation capacity resulting in non-viable offspring. Therefore, the standard breeding practice for nude mice is to mate a homozygous male and heterozygous female. One half of the resulting offspring are homozygous for a recessive Foxnl^{nu} mutation, and therefore immunodeficient, and lacking a hair coat. The other half of the offspring are heterozygous for a recessive Foxnl^{nu} mutation, and thus immunocompetent, and haired. As such, half of the animals produced by the standard breeding practice of nude mice do not fit the immunodeficiency requirements for xenograft studies and, therefore, are unusable for these studies. Similar breeding challenges also exist for NIH III mice. Thus, the breeding of athymic nude mice and NIH III mice is inefficient as one half of the progeny are generally not used in medical research. As a result, there remains a need for an efficient breeding protocol to produce immunodeficient mice.

### SUMMARY

The present invention is based, in part, upon the discovery that a mouse homozygous for a recessive hairless hr allele (Hr^{hr}) can be bred with an immunodeficient mouse that is homozygous for a mutation that disrupts differentiation of both B lymphocyte progenitor cells and T lymphocyte progenitor cells to produce a hairless, immunodeficient mouse that is homozygous at both the hairless allele and the mutation resulting in the B-cell deficiency and T-cell deficiency. The hairless, immunodeficient mice described herein offer an advantage over both the athymic mouse model and NIH III mouse model because these mice can be maintained as homozygous breeding pairs. As a result, all of the progeny from these breeding pairs can be used for xenograft studies and, therefore, the number of animals that cannot be used for xenograft studies is significantly reduced. In addition, the hairless, immunodeficient mice described herein comprise both a B-cell deficiency and a T-cell deficiency and, therefore, are more severely immunocompromised than athymic nude mice allowing a higher percentage of engraftment, increased tumor growth rate, and less tumor regression. The hairless mice are particularly useful as the xenografts can usually be identified visually without the need for palpation. In contrast, when the animals have a hair coat, the xenografts usually need to be identified by touch.

The present invention discloses a hairless, immunodeficient mouse that is homozygous for a recessive hr allele (Hr^{hr}) and further comprises a mutation that disrupts differentiation of both B lymphocyte progenitor cells and T lymphocyte progenitor cells. In certain embodiments, the hairless, immunodeficient mouse is homozygous for the mutation that disrupts differentiation of both B lymphocyte progenitor cells and T lymphocyte progenitor cells. An exemplary hairless, immunodeficient mouse is a mouse homozygous for a recessive scid allele (Prkdc^{scid}). Other exemplary hairless, immunodeficient mice are homozygous for a recessive beige allele (Lyst^{bg}) or homozygous for a mutated Rag allele.

The present invention also provides methods for producing a hairless, immunodeficient mouse comprising breeding a first mouse strain homozygous for a recessive hairless (hr) allele (Hr^{hr}) with a second, different mouse strain homozygous for a mutation that disrupts differentiation of both B lymphocyte progenitor cells and T lymphocyte progenitor cells. Progeny from the F1 cross are heterozygous for both the hr allele and the mutation disrupting differentiation of both B lymphocyte progenitor cells and T lymphocyte progenitor cells. The heterozygous progeny then are intercrossed and homozygous hr progeny are selected. Hairless homozygous hr progeny from the first intercross are either homozygous or heterozygous for the mutation disrupting differentiation of both B lymphocyte progenitor cells and T lymphocyte progenitor cells, and are intercrossed to obtain progeny homozygous at both the hr allele and the mutation disrupting differentiation of both B lymphocyte progenitor cells and T lymphocyte progenitor cells. Homozygosity at both mutations may be confirmed by genetic testing methods well known in the art.

These and other aspects and advantages of the invention will become apparent upon consideration of the following detailed description and claims.

### DETAILED DESCRIPTION

The present invention discloses hairless, immunodeficient mouse models that are homozygous for a recessive hr allele (Hr^{hr}) and further comprise a mutation that results in both a B-cell deficiency and a T-cell deficiency. The hairless, immunodeficient mouse models described herein offer a significant advantage over the athymic nude mouse model and the NIH III mouse model because they can be maintained using homozygous breeding pairs. As a result, all of the progeny obtained from the homozygous breeding pairs are hairless and comprise both a B-cell deficiency and a T-cell deficiency. The ability to breed and maintain hairless, immunodeficient mouse colonies significantly reduces the number of animals, which are produced and not useable in medical research for their intended purpose. Further, the hairless, immunodeficient mouse models described herein are more severely immunocompromised than the athymic nude mice. The greater immunodeficiency of the mouse models of the invention allow for better growth of xenogenic tumor lines. Furthermore, hairless mice also allow rapid assessment of cutaneous and subcutaneous tissue and tumor grafts.

In one aspect, the invention provides a hairless, immunodeficient mouse having a hairless (hr) allele and further comprising a mutation that disrupts differentiation of both B lymphocyte progenitor cells and T lymphocyte progenitor cells. In certain embodiments, the mutation that disrupts differentiation of both B lymphocyte progenitor cells and T lymphocyte progenitor cells is a homozygous mutation.

In one embodiment, the hairless, immunodeficient mouse is homozygous for a recessive hr allele (Hr^{hr}) and homozygous for a recessive scid allele (Prkdc^{scid}). Mice homozygous for both the hairless and scid alleles are phenotypically characterized as hairless, B-cell deficient, and T-cell deficient. Hairless, scid mice are further characterized as maintaining normal natural killer cells, macrophages, and granulocytes, and possess lymph nodes and a thymus. Under certain circumstances, it is desirable to produce mice homozygous for a recessive hr allele (Hr^{hr}) and homozygous for a scid allele (Prkdc^{scid}) that are also homozygous for a recessive beige allele (Lyst^{bg}). Mice homozygous for the hairless, scid, and beige alleles are phenotypically characterized as hairless, B-cell deficient, T-cell deficient, have granulocytes that are defective and have reduced activity and a severe deficiency of natural killer cells.

In another embodiment, the hairless, immunodeficient mouse is homozygous for a recessive hr allele (Hr^{hr}) and homozygous for a mutated Rag allele. The mutated Rag allele can be a mutated Rag1 allele or a mutated Rag2 allele. The mutated Rag1 and Rag 2 alleles can be a homozygous mutation, and can be homozygous for a targeted null mutation at the either or both Rag loci. Mice homozygous for both the hairless allele and a mutated Rag allele are phenotypically characterized as hairless, B-cell deficient, and T-cell deficient.

In another aspect, the invention provides methods for producing a hairless, immunodeficient mouse. The method comprises the steps of (a) crossing a first mouse strain homozygous for a recessive hr allele with a second, different mouse strain homozygous for a mutation that disrupts differentiation of both B lymphocyte progenitor cells and T lymphocyte progenitor cells to produce progeny heterozygous for both mutations; (b) intercrossing the heterozygous progeny produced by step (a); (c) selecting hairless progeny produced by step (b) homozygous for the hr allele; and (d) intercrossing hairless mice homozygous or heterozygous for the mutation resulting in a B-cell deficiency and T-cell deficiency to obtain progeny homozygous for both mutations.

Hairless progeny homozygous for the recessive hr allele can be selected based on phenotypic characterization. Mice homozygous for the hr allele develop a hair coat after birth, but lose their hair coat within one-month of birth, for example, within about 20 days of birth, within about 25 days of birth, or within about 30 days (about one-month) of birth. Mice homozygous for the recessive hr allele may be selected based on the loss of their hair coat prior to or at weaning or within one-month of birth. Mice heterozygous for the recessive hr allele do not lose their hair coat. Hairless progeny homozygous for the recessive hr allele may also be selected by genotypic testing methods well known in the art, including, for example, tail biopsies and ear clippings.

In some embodiments, the method for producing a hairless, immunodeficient mouse may further comprise the step of determining the zygosity of the mutation disrupting the differentiation of both B lymphocyte progenitor cells and T lymphocyte progenitor cells in the hairless progeny selected in step (c). Zygosity of the mutation resulting in both a B-cell deficiency and T-cell deficiency may be determined by genetic testing methods well known in the art, including, for example, tail biopsies and ear clippings.

In another embodiment, the method for producing a hairless, immunodeficient mouse may further comprise the step of, after step (d), confirming that the progeny are homozygous for the recessive hr allele and homozygous for the mutation disrupting differentiation of both B lymphocyte progenitor cells and T lymphocyte progenitor cells. Zygosity of both mutations may be confirmed by genetic testing methods well known in the art, including, for example, tail biopsies and ear clippings.

Exemplary hairless mice that can be used to produce the hairless immunodeficient mice of the invention are homozygous for the recessive hr allele and are characterized as euthymic and immunocompetent. One such mouse useful in making the hairless models described herein is the Crl:SKH1-Hr^{hr} mouse strain that can be obtained from Charles River Laboratories, Wilmington, Mass.

Exemplary mice having one or more mutations that disrupt the differentiation of both B and T lymphocyte progenitor cells that can be used to produce the immunodeficient mice of the invention include mice having one or more mutations in the scid allele, the beige allele, and the Rag allele. Mice having one or more mutations in the scid allele, the beige allele, and the Rag allele may be maintained as inbred or outbred lines.

In one embodiment, the second mouse strain homozygous for a mutation that disrupts differentiation of both B lymphocyte progenitor cells and T lymphocyte progenitor cells is a mouse strain homozygous for a recessive scid allele (Prkdc^{scid}). Scid mice possess an autosomal recessive mutation in the Prkdc gene and are characterized as having a severe combined immunodeficiency disrupting differentiation of both B lymphocyte progenitor cells and T lymphocyte progenitor cells. Scid mice lack dendritic Thy-1+ epidermal cells and this deficiency results in their inability to produce antibodies and to reject allogenic and xenogenic tissue and tumor grafts. Scid mice are further characterized as maintaining normal natural killer cells, macrophages, and granulocytes, and possess lymph nodes and a thymus. Scid mice, for example, the Crl:HA-Prkdc^{scid} mouse strain, can be obtained from Charles River Laboratories, Wilmington, Mass. In another embodiment, the mouse strain homozygous for a recessive scid allele is additionally homozygous for a recessive beige allele (Lyst^{bg}). SCID Beige mice possess autosomal recessive mutations in both the Prkdc gene and the Lyst gene. In addition to lacking functional B lymphocytes and T lymphocytes, beige mice also lack natural killer (NK) cells and are characterized as B-cell deficient, T-cell deficient, and NK cell deficient. Beige mice, for example, the CB17.B6-Prkdc^{scid} Lys mouse strain, can be obtained from Charles River Laboratories, Wilmington, Mass.

In another embodiment, the second mouse strain homozygous for a mutation that disrupts differentiation of both B lymphocyte progenitor cells and T lymphocyte progenitor cells is a mouse strain homozygous for a mutated Rag allele. The mutated Rag allele can be a mutated Rag1 allele or a mutated Rag2 allele. The Rag1 or 2 mutation can be a targeted null mutation at the Rag1 or 2 allele. The mutated Rag1 or 2 allele can be homozygous for the mutation, and these mice produce no mature T cells or B cells. The mutated Rag] phenotype can be described as a “non-leaky” severe combined immune deficiency because they do not produce B cells and IgM unlike homozygous Prkdc^{scid} mice that sometimes produce very small numbers of B cells and IgM. They have no CD3⁺ or T cell receptor (TCR) alpha-beta positive cells and the thymus of the mutant mice contain 15 to 130 times fewer cells than heterozygous or wild type siblings. The thymocytes are CD8⁻CD4⁻ and may be IL-2 receptor positive. The spleen and bone marrow from homozygous mutant Rag1 mice do not contain any IgM or IgD staining cells, indicating an absence of mature B cells. These and other data suggest that B cell and T cell development has been arrested at an early stage in these mice. Mice homozygous for a mutated Rag2 allele exhibit arrested development of T cell and B cell maturation at the CD4⁻CD8⁻ thymocyte or B220⁺/CD43⁺pro-B cell stage due to inability to undergo V(D)J recombination. Mouse strains mutated for the Rag1 or 2 allele are commercially available.

A hairless, immunodeficient mouse homozygous for a recessive hr allele (Hr^{hr}) and homozygous for a recessive scid allele (Prkdc^{scid}) can be produced by crossing a hairless mouse with a scid mouse. An exemplary hairless mouse strain is Crl:SKH1-Hr^{hr} and an exemplary scid mouse strain is Crl:HA-Prkdc^{scid}. The progeny resulting from the F1 cross are heterozygous for the recessive hr allele and the recessive scid allele. The heterozygous progeny then can be intercrossed to begin returning the mutations to the homozygous state. Hairless F2 mice then are selected based on phenotype, i.e., the loss of their hair coat. Tail biopsies can be collected at weaning from the hairless mice and submitted for genetic testing to determine the zygosity of the scid mutation. Mice that are either homozygous or heterozygous for the scid mutation can be selected for mating to produce F3 progeny. All mice progeny are hairless, and tail biopsies are taken from all mice to determine zygosity of the scid allele. Double homozygous mice are selected to build a mouse colony of hairless-scid mice.

Under certain circumstances, it is beneficial for the scid mouse to also be homozygous for a recessive beige allele (Lyst^{bg}). An exemplary scid-beige mouse strain is CB17.B6-Prkdc^{scid} Lyst^{bg}. The animals can be produced essentially as described above. The F2 progeny are selected based on phenotype, i.e., the loss of their hair coat. Tail biopsies are collected at weaning from the hairless mice and submitted for genetic testing to determine the zygosity of the scid allele. Blood smears can be used to identify animals homozygous for the beige mutation. Mice that are either homozygous or heterozygous for the scid mutation and homozygous for the beige mutation are selected for mating to produce F3 progeny. Triple homozygous mice are selected to build a colony of hairless scid-beige mice.

In another embodiment, hairless, immunodeficient mouse homozygous for a recessive hr allele (Hr^{hr}) and homozygous for a mutated Rag allele can be produced by crossing a hairless mouse with a mouse homozygous for a mutated Rag allele. Exemplary mouse strains comprising a mutated Rag allele include the commercially available mouse strains B6.129S7-Rag1^{tmlMom}/J and 129S6/SvEvTac-Rag2^{tmlFwa}. The progeny resulting from the F1 cross is heterozygous for the recessive hr allele and the mutated Rag allele. As described above, the heterozygous progeny then are intercrossed to begin to return the mutations to the homozygous states and hairless F2 mice are selected based on phenotype, i.e., the loss of their hair coat. Tail biopsies are collected at weaning from the hairless mice and submitted for genetic testing to determine the zygosity of the mutated Rag allele. Mice that are either homozygous or heterozygous for the mutated Rag allele are selected for mating to produce F3 progeny. The F3 progeny can be genotyped to determine the zygosity of the mutated Rag allele. Double homozygous mice are selected to build a mouse colony of hairless-Rag mice.

The hairless, immunodeficient mouse models described herein can be maintained as a mouse colony by breeding male and female mice each of which are mouse homozygous for a recessive hr allele (Hr^{hr}) and homozygous for a mutation that disrupts differentiation of both B lymphocyte progenitor cells and T lymphocyte progenitor cells. The ability to breed hairless, immunodeficient male mice with hairless, immunodeficient female mice produces progeny that are all hairless and immunodeficient.

The hairless, immunodeficient mouse models described herein comprise both a B-cell deficiency and a T-cell deficiency. As a result, these mouse models are susceptible to infection from a variety of organisms, which would not otherwise pose a threat to an immunocompetent mouse. In addition to mortality and/or clinical illness, such an infection may induce responses in the remaining host-defense mechanisms of the hairless, immunodeficient mouse that inhibit growth of implanted tumors. Further, xenograft studies in the hairless, immunodeficient mice described herein may include the use of chemotherapeutic compounds. Chemotherapeutic compounds may suppress the production of leukocytes, such as neutrophils, which play key role in the rapid immune response to bacterial challenge, emphasizing the need for mice free of specified bacteria and other infectious organisms, examples of which are described herein. Thus, hairless, immunodeficient mouse models described herein preferably are bred, housed, and transported in a manner that excludes pathogens, for example, pathogenic bacteria, fungi, parasites and viruses. Accordingly, the animals are bred, housed, transported, and otherwise maintained under conditions to be pathogen free.

In certain embodiments, the hairless, immunodeficient mouse models described herein are free of pathogens (for example, pathogenic bacteria, viruses, fungi or parasites) that cause clinical disease or subclinical infections. In one embodiment, the hairless, immunodeficient mouse models described herein are free from viruses that may cause clinical disease or subclinical infections, including the viruses selected from the group consisting of mouse rotavirus (EDIM), Sendai (SEND), mouse cytomegalovirus (MCMV), mouse hepatitis virus (MHV), mouse norovirus (MNV), mouse parvoviruses (MPV1-4), minute virus of mice (MVM), mouse thymic virus (MTLV), Theiler's murine encephalomyelitis virus (TMEV), K virus (K), mousepox (Ectromelia), lactate dehydrogenase-elevating virus (LDV), Hantaviruses, pneumonia virus of mice (PVM), reovirus types 1 and 3 (REO), mouse adenovirus (MAV-1 and MAV-2), lymphocytic choriomeningitis virus (LCMV), and polyoma virus (POLY).

In another embodiment, the hairless, immunodeficient mouse models described herein are free from bacteria that may cause clinical disease or subclinical infections, including the bacteria selected from the group consisting of Salmonella, CAR bacillus, Helicobacter (including all Heliobacter species), Clostridium piliforme, Corynebacterium kutscheri, Corynebacterium bovis, Citrobacter rodentium, Mycoplasma pulmonis and other species, Streptobacillus moniliformis, Bordetella bronchiseptica, Beta-hemolytic streptococci, Streptococcus pneumoniae, Staphylococcus aureus, other coagulase-positive staphylococci, Pasteurella pneumotropica, Pseudomonas (all species), and Klebsiella (all species).

In another embodiment, the hairless, immunodeficient mouse models described herein are free from fungi that may cause clinical disease or subclinical infections, including the fungi selected from the group consisting of Encephalitozoon cuniculi (ECUN) and Pneumocystis.

In another embodiment, the hairless, immunodeficient mouse models described herein are free from parasites that may cause clinical disease or subclinical infections, including the parasites selected from the group consisting of external and internal multicellular and unicellular parasites.

In certain embodiments, the hairless, immunodeficient mouse models are tested for a panel of infectious organisms that may cause clinical disease or subclinical infections including, for example, mouse rotavirus (EDIM), Sendai (SEND), mouse cytomegalovirus (MCMV), mouse hepatitis virus (MHV), pneumonia virus of mice (PVM), Encephalitozoon cuniculi (ECUN), mouse parvoviruses (MPV1-4), minute virus of mice (MVM), K virus (K), lactate dehydrogenase-elevating virus (LDV), Hantaviruses, reovirus types 1 and 3 (REO), mouse adenovirus (MAV-1 and MAV-2), lymphocytic choriomeningitis virus (LCMV), polyoma virus (POLY), Theiler's murine encephalomyelitis virus (TMEV, GD-7), Ectromelia (Ectro), CAR bacillus, Clostridium piliforme, Corynebacterium kutscheri, Mycoplasma pulmonis, Bordetella bronchiseptica, Citrobacter rodentium, Helicobacter hepaticus, Salmonella species, Streptobacillus moniliformis, Pneumocystis carinii, β-hemolytic Streptococcus species, coagulase (+) Staphylococcus species, Corynebacterium bovis, Pasteurella pneumotropica, Streptococcus pneumoniae, Mycoplasma species, Helicobacter bilis, Helicobacter species, coagulase (−) Staphylococcus species, non-β-hemolytic Streptococcus species, Klebsiella species, Pseudomonas aeruginosa, Corynebacterium species, and Bacillus species.

### Exemplification

The following Examples discuss the production and characterization of the hairless, immunodeficient mouse models described herein.

### EXAMPLE 1

### Hairless-Scid Mouse Model

A hairless, immunodeficient mouse homozygous for a recessive hr allele (Hr^{hr}) and homozygous for a recessive scid allele (Prkdc^{scid}) was generated by crossing a male hairless mouse (strain Crl:SKH1-Hr^{hr}) with a female scid mouse (strain Crl:HA-Prkdc^{scid}). Prior to crossing the two lines, each one was maintained as a genetically outbred stock. The progeny resulting from the F1 cross were heterozygous for the recessive hr allele and the recessive scid allele. The heterozygous progeny then were intercrossed to begin returning the mutations to the homozygous state. Hairless F2 mice were selected based on phenotype, i.e., the loss of their hair coat. Tail biopsies were collected at weaning from the hairless mice and submitted for genetic testing to determine the zygosity of the scid mutation. Mice that were either homozygous or heterozygous for the scid mutation were selected for mating to produce F3 progeny. All mice produced were hairless, and tail biopsies were taken from all mice to determine zygosity of the scid allele. Double homozygous mice were selected to build a mouse colony of hairless-scid mice.

### EXAMPLE 2

### Hairless-Rag1 Mouse Model

A hairless, immunodeficient mouse homozygous for a recessive hr allele (Hr^{hr}) and homozygous for a mutated Rag1 allele (Rag1^{tmlMom}) is generated by crossing a male hairless mouse (strain Crl:SKH1-Hr^{hr}) with a female mouse homozygous for a mutated Rag1 allele (strain B6.129S7-Rag1^{tmlMom}/J). The progeny resulting from the F1 cross is heterozygous for the recessive hr allele and the mutated Rag1 allele. The heterozygous progeny then are intercrossed to begin to return the mutations to the homozygous states. Hairless F2 mice are selected based on phenotype, i.e., the loss of their hair coat. Tail biopsies are collected at weaning from the hairless mice and submitted for genetic testing to determine the zygosity of the mutated Rag1 allele. Mice that are either homozygous or heterozygous for the mutated Rag1 allele are selected for mating to produce F3 progeny. It is expected that all F3 progeny are hairless and they are genotyped to determine the zygosity of the mutated Rag1 allele. Double homozygous mice are selected to build a mouse colony of hairless-Rag1 mice.

### EXAMPLE 3

### Hairless-Rag2 Mouse Model

A hairless, immunodeficient mouse homozygous for a recessive hr allele (Hr^{hr}) and homozygous for a targeted null mutation at the Rag2 allele are generated by crossing a male hairless mouse (strain Crl:SKH1-Hr^{hr}) with a female mouse homozygous for a targeted null mutation at the Rag2 allele (strain 129S6/SvEvTac-Rag2^{tmlFwa}). The progeny resulting from the F1 cross are heterozygous for the recessive hr allele and the null mutation at the Rag2 allele. The heterozygous progeny then are intercrossed to begin to return the mutations to the homozygous states. Hairless F2 mice are selected based on phenotype, i.e., the loss of their hair coat. Tail biopsies are collected at weaning from the hairless mice and submitted for genetic testing to determine the zygosity of the null Rag2 allele. Mice that are either homozygous or heterozygous for the null Rag2 allele are selected for mating to produce F3 progeny. It is expected that all F3 progeny are hairless and they are genotyped to determine the zygosity of the null Rag2 allele. Double homozygous mice are selected to build a mouse colony of hairless-Rag2 mice.

### Incorporation by Reference

The entire disclosure of each of the following patent documents and scientific reference are incorporated by reference for all purposes: U.S. Pat. Nos. 5,583,278; 5,859,307; and Bosma, G. C. et al., Nature 301:527-530 (1983). In case of conflict, the present application, including any definitions, will control.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A hairless, immunodeficient mouse having a hr allele and both a B-cell deficiency and a T-cell deficiency, wherein the mouse is homozygous for the recessive hr allele (Hr^{hr}) and further comprises a mutation that disrupts differentiation of both B lymphocyte progenitor cells and T lymphocyte progenitor cells.

2. The mouse of claim 1, wherein the mouse is homozygous for a recessive scid allele (Prkdc^{scid}).

3. The mouse of claim 1, wherein the mouse is homozygous for a mutated Rag allele.

4. The mouse of claim 3, wherein the mouse is homozygous for a Rag1 mutation.

5. The mouse of claim 3, wherein the mouse is homozygous for a Rag2 mutation.

6. The mouse of claim 1, wherein the mouse has hair after birth, but then loses its hair within one month after birth.

7. A method for producing a hairless, immunodeficient mouse, the method comprising the steps of:
(a) crossing a first mouse strain homozygous for a recessive hr allele with a second, different mouse strain homozygous for a recessive scid allele to produce progeny heterozygous for both the hr allele and the scid allele;
(b) intercrossing the heterozygous progeny produced by step (a);
(c) selecting hairless progeny produced by step (b) homozygous for the hr allele; and
(d) intercrossing hairless mice homozygous or heterozygous for the scid allele to obtain progeny homozygous for both the hr allele and the scid allele.

8. The method of claim 7, further comprising the step of determining the zygosity of the scid allele in the hairless progeny selected in step (c).

9. The method of claim 8, wherein the zygosity of the scid allele is determined by genetic testing.

10. The method of claim 7, wherein, in step (a), the first mouse strain is a Crl:SKH1-Hr^{hr} mouse.

11. The method of claim 7, wherein, in step (a), the second mouse strain is a Crl:HA-Prkdc^{scid} mouse.

12. The method of claim 7, further comprising the step of, after step (d), confirming that the progeny are homozygous for the recessive hr allele and homozygous for the recessive scid allele.

13. A colony of mice of claim 1.

14. A colony of mice produced by the method of claim 7.
